(19) United States
(12) Patent Application Publication
(10) Pub. No.: US 2011/0082339 A1
(43) Pub. Date: Apr. 7, 2011

(54) METHOD AND APPARATUS FOR STABILIZING AN ABDOMINAL WALL DURING A LAPAROSCOPIC INCISION

(76) Inventor: Glynn J. Elliott, III, Lincolnwood, IL (US)

(21) Appl. No.: 12/572,848

(22) Filed: Oct. 2, 2009

(51) Int. Cl. A61B 1/32; A44B 13/00

(52) U.S. Cl. 600/204; 24/592.1

(57) ABSTRACT

An apparatus and method for laparoscopic surgery provides a stabilizing tool using having movable latches. In one embodiment, the stabilizing tool includes a body portion, a moving mechanism and a latching mechanism. The moving mechanism enables a user to extend the movable latches outwardly to an extended position and retract the movable latches inwardly to a retracted position.

### COPYRIGHT NOTICE

A portion of the disclosure of this patent document contains material which is subject to copyright protection. The copyright owner has no objection to the photocopy reproduction of the patent document or the patent disclosure in exactly the form it appears in the Patent and Trademark Office patent file or records, but otherwise reserves all copyright rights whatsoever.

### TECHNICAL FIELD

The present invention relates in general to laparoscopic surgery and more specifically to stabilizing an abdominal wall during a laparoscopic incision.

### BACKGROUND

Laparoscopic surgery is used to perform surgical procedures that have traditionally been performed using open surgical procedures. The patient benefits of having a laparoscopic procedure include reduced trauma to patient tissue, smaller scars, less post-surgical pain, and faster recovery and return to regular activity levels.

Laparoscopic surgery typically involves the use of a trocar. A trocar is surgical instrument which is inserted into the body to allow exchange of other surgical instruments such as, for example, a cannula. In one preferred method, the surgeon inserts the trocar into the body using a blind puncture. The blind insertion uses a technique known as a controlled jab. The force required to puncture the patient varies from patient to patient and varies depending on the sharpness of the trocar blade. The surgeon must apply sufficient force to penetrate the abdomen, while using suitable control to stop the trocar from moving when penetrated. Many surgeons prefer to elevate the abdominal wall before the blind puncture to reduce the injury of internal organs. To elevate the abdominal wall, a laparoscopist grabs onto the abdominal wall with their hand and lifts upwardly while the initial incision is made. However, this method of elevating a patient's abdominal wall by grabbing the patient's abdomen provides risks. For example, the laparoscopist holding onto the abdomen during the initial incision may not control the abdomen as necessary for the initial incision, which may cause severe injuries for the patient.

### SUMMARY

One embodiment of the preset disclose includes a fascia stabilizing apparatus including a housing having a distal end, a latching mechanism including a first latch and a second latch wherein the latching mechanism is positioned at the distal end of the housing, and a graspable member operatively coupled to the first larch and the second latch of the latching mechanism. The first latch has a first hook portion and the second latch has a second hook portion. The first latch and the second latch are moveable between a first position which distances the first hook portion and the second hook portion from an elongated axis of the housing by a first distance and a second position which distances the first hook portion and the second hook portion from the elongated axis by a second greater distance. The graspable member is operable to cause the first latch and the second latch to move between the first position and the second position.

### BRIEF DESCRIPTION OF THE FIGURES

FIG. 1 is a perspective view of an example of a stabilizing apparatus with latches in a retracted position.

FIG. 2 is a cross-sectional perspective view of the stabilizing apparatus of FIG. 1, taken substantially along line 1-1 of FIG. 1, illustrating the latches in the retracted position.

FIG. 3 is a cross-sectional side view of the stabilizing apparatus of FIG. 1, taken substantially along line 1-1 of FIG. 1, illustrating the latches in the extended position.

FIG. 4 is a perspective view illustrating one example of a movable latch.

FIG. 5 is a schematic diagram of one example of the stabilizing apparatus having an electronic configuration, illustrating a controller, a logic circuit, input devices and output devices.

### DETAILED DESCRIPTION OF EXEMPLARY EMBODIMENTS

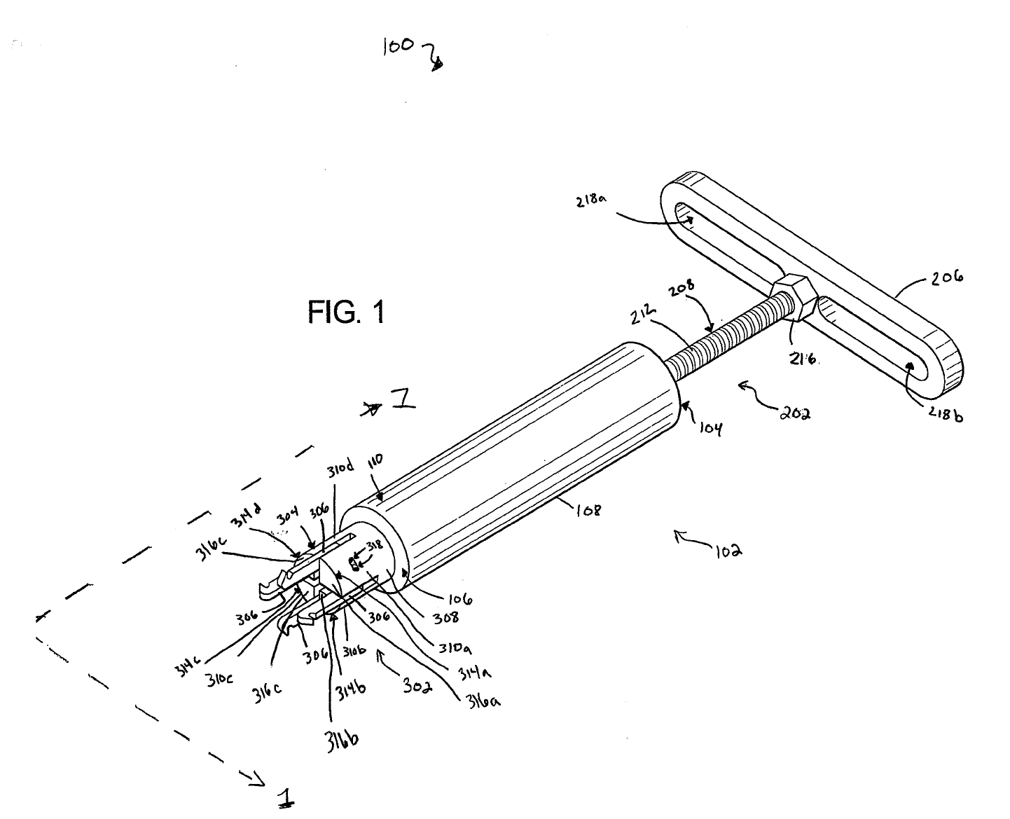

Referring now to FIGS. 1 through 3, the stabilizing apparatus 100 includes: (a) an elongated body portion generally indicated at 102; (b) a moving mechanism generally indicated at 202; and (c) a latching mechanism generally indicated at 302.

In this example, the body portion 102 includes: (a) a proximal end 104; (b) a distal end 106; (c) a tubular structure 108; and (d) a cylindrical extension portion 110. The body portion has a generally circular cross-section as best shown in FIG. 1. The body portion 102 defines an opening 112a at the proximal end 104 of the body portion 102, and an opening 112b at the distal end 106 of the body portion 102. In this example, the tubular structure 108 has a threaded interior surface 114 and defines an interior space 116. The opening 112b extends through the cylindrical extension 110. The latching mechanism 302, described in more detail below, is partially inserted through the opening 112b. The cylindrical extension 110 has an inside surface 118. In this example, a portion of the inside surface is threaded as shown at 120. The cylindrical extension 110 and the tubular structure 108 define an interior space 122.

In this example, the moving mechanism 202 includes: (a) a graspable member 206; and (b) a shaft 208 connected to a cylinder 210. In this example, the shaft 208 has a threaded surface 212.

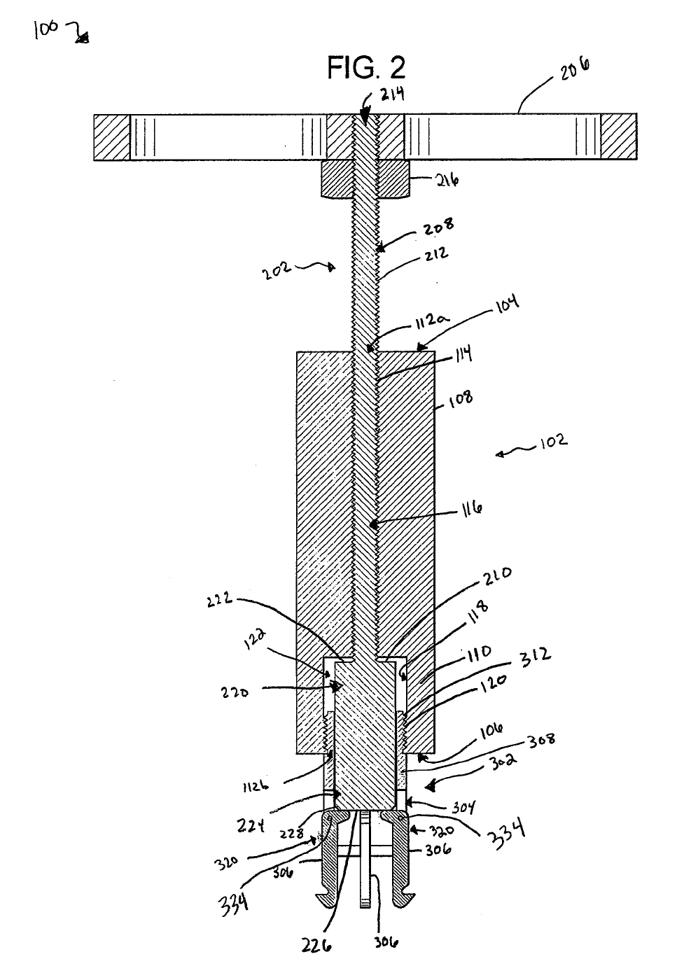

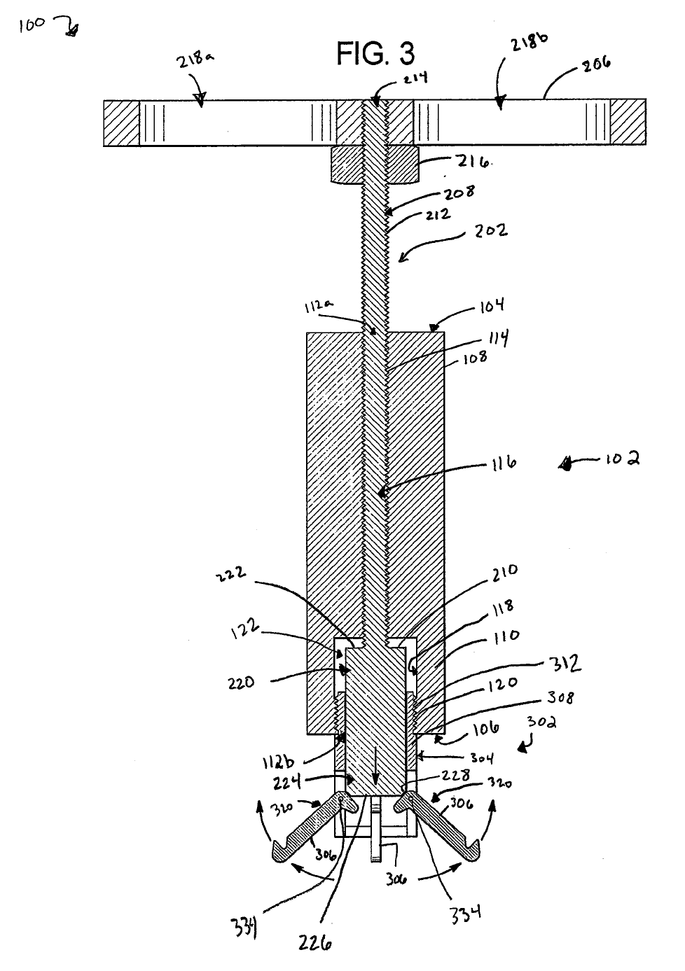

The graspable member 206 defines a threaded opening 214 which matingly engages the threaded shaft 208 as best shown in FIGS. 2 and 3. In this example, a nut 216 is engaged with the threaded shaft 208 to prevent the graspable member 206 from moving in a downward direction along the threaded shaft 208. In this example, the graspable member 206 also defines openings 218a and 218b as best shown in FIG. 1.

The threaded shaft 208 is rotatably disposed within the tubular structure 108. The threaded shaft 208 matingly engages the threaded interior surface 114 of the tubular structure 108 as best shown in FIGS. 2 and 3. The threaded shaft 208 and the body portion 102 are coaxial and share a longitudinal axis. As described in more detail below, the body portion 102 and the threaded shaft 208 can be rotated about the longitudinal axis to extend the latching mechanism.

The cylinder 210 has: (a) a top portion 220 having a top surface 222; and (b) a bottom portion 224 having a bottom surface 226 and an angled surface 228. The top portion 220 is connected to the threaded shaft 208 as best shown in FIGS. 2 and 3.

The latching mechanism 302 includes: (a) a latch holder 304; and (b) a plurality of movable latches 306 operatively connected to the latch holder 304.

The latch holder 304 includes a body having: (a) a tubular portion 308; and (b) a plurality of curved extensions 310a, 310b, 310c and 310d which extend longitudinally from the tubular portion 308; and (c) a plurality of bottom portions 316a, 316b, 316c and 316d which extend laterally from the distal ends of each of the curved extensions 310a, 310b, 310c and 310d, respectively. The tubular portion 308 includes an outer threaded surface portion 312. The outer threaded surface portion 312 matingly engages the threaded surface 120 of the cylindrical extension 110 as best shown in FIGS. 2 and 3.

Each curved extensions 310a, 310b, 310c and 310d has a distal end 314a, 314b, 314c and 314d, respectively. In this example, each bottom 316a, 316b, 316c and 316d extends from each curved extension 310a, 310b, 310c and 310d, respectively, at a ninety degree angle. Each curved extension 310a-d defines a plurality of openings 318a-d, respectively. Each opening is configured to receive a pin 334 discussed in more detail below.

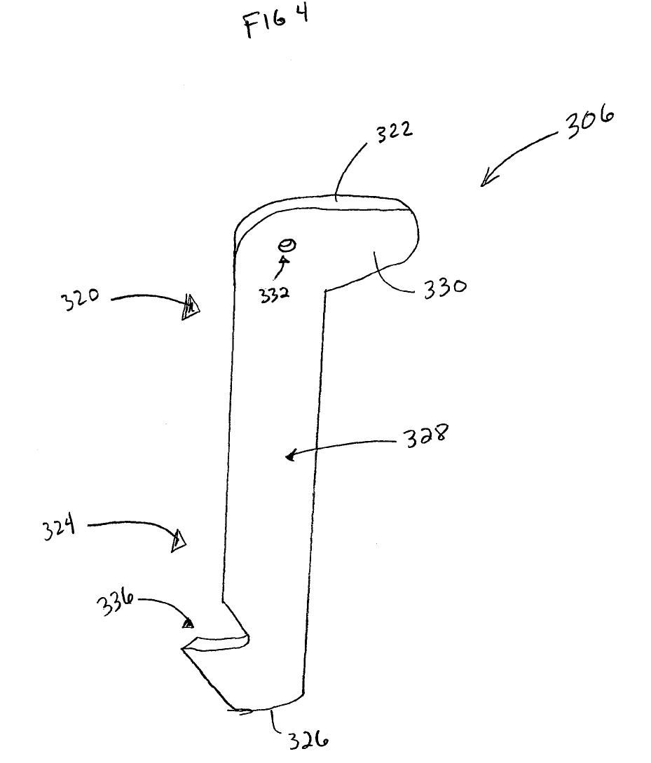

As best shown in FIG. 4, the plurality of movable latches 306 each include: (a) a top portion 320 having a top surface 322; and (b) a bottom portion 324 having a bottom surface 326; and (c) an elongated body 328. The top portion 320 includes an extension 330.

The movable latches 306 are pivotally attached to the latch holder 304. The top portion 320 of each latch defines an opening 332 which is shaped to receive a pin 334. Each pin 334 is positioned through an opening 318 of the latch holder 304, and though the latch opening 332 of the movable latch 306 to pivotally attach the latch 306 to the latch holder 304. The bottom portion 320 of the movable latch includes a hook portion 336.

In operation, generally, a user positions the hook portion of the latching mechanism, in a retracted position, inside a patient's navel. The moving mechanism causes the plurality of movable latches to move from the retracted position to an extended position. As the plurality of movable latches move to the extended position, the hook portion of the latches engage the walls of the navel. This engagement allows the user to stabilize the patient's abdominal wall.

More specifically, when the user positions the hook portion 328 inside the navel, the plurality of latches 306 are in the retracted position. When the plurality of latches 306 are in the retracted position, the cylinder 210 is in an upward position as best shown in FIG. 2. When the cylinder 210 is in the upward position, the bottom surface 226 of the cylinder 210 is in contact with the top surfaces 322 of the plurality of movable latches 306. When the cylinder 210 is in the upward position, the angled surface 228 of the cylinder is not in contact with the top surfaces 322 of the plurality of latches 306.

The cylinder 210 moves from the upward position to a downward position when the user turns the graspable member 206 in a clockwise rotation as best shown in FIG. 3. As the cylinder 210 moves from the upward direction to the downward direction, the plurality of movable latches 306 begin to pivot outwardly into the extended position as best shown in FIG. 3. When the cylinder 210 moves from the upward position in the downward direction, the top surfaces 322 of the plurality of movable latches 306 become in contact with the angled surface 228 of the cylinder 210. When the plurality of latches 306 are in the extended position, the angled surface 228 of the cylinder 210 prevents the movable latches 306 from moving to the retracted position. The top surfaces 322 of the movable latches 306 are not in contact with the bottom surface 226 of the cylinder 210 when the movable latches 306 are in the extended position.

As the movable latches 306 are spread away from one another, the hook portions 336 engage the patient's navel which allows the user to stabilize an abdominal wall of a patient. The user is then enabled to pull in the upward direction thereby elevating the abdominal wall.

In one example, the body portion 102 can include an elongated grip configured to provide an ergonomic grip for the operator of the stabilizing instrument.

In one example, the body portion defines an opening so that an auxiliary surgical instrument can be inserted. In one example, the fascia stabilizing apparatus may be hollow to allow a needle, such as a veress needle, to pass though the fascia stabilizing apparatus. In one example, the auxiliary surgical instrument may be an endoscope that includes a camera which enables the user to observe the inside of the patient's body. In another example, the user may introduce cutting instruments in the opening so that the user may have more control making a cut.

In one example, where the fascia stabilizing apparatus allows an auxiliary surgical instrument to be inserted, the fascia stabilizing apparatus is configured such that it could be disassembled. In one example, after a needle is passed through the fascia stabilizing apparatus and after insulflation has occurred in the abdomen, the fascia stabilizing apparatus can be disassembled so that the fascia stabilizing apparatus can be removed while the needle remains inserted.

In one alternative example, the threaded shaft and the body portion are not coaxial.

In one embodiment, the stabilizing apparatus includes a light attached to the end of the end portion of the stabilizing apparatus which enhances visibility for the user.

In one example, the threaded shaft 208 is solid. In another example, the threaded shaft 208 is tubular. In one example, the shaft 208 and the cylinder 222 are made up of one solid piece.

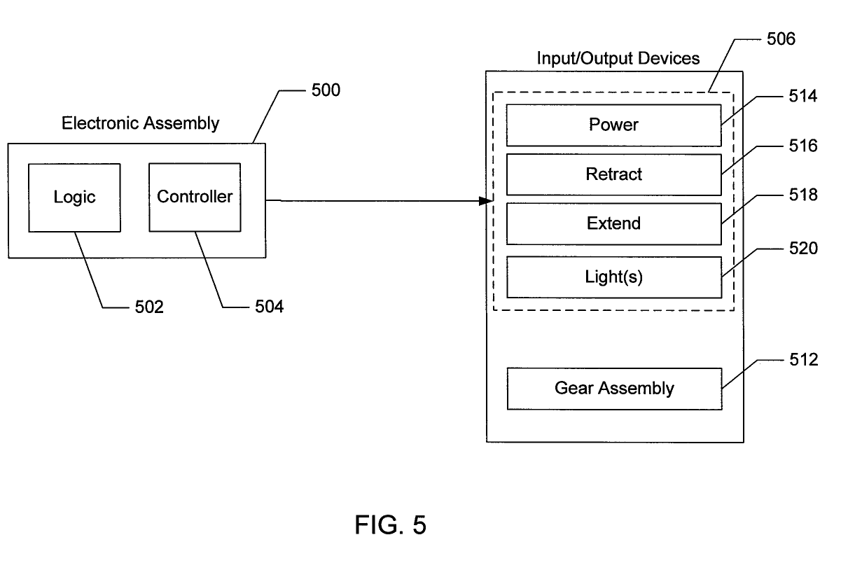

Referring to FIG. 5, in one example, the moving mechanism includes an electronic assembly 500 which has a controller 502 coupled to logic 504. In one example, the controller 502 includes one or more digital processors (not shown), such as a digital microprocessor or a micro-controller based platform. In one example, the controller 502 includes one or more analog control units (not shown) such as a suitable integrated circuit or one or more application-specific integrated circuits (ASIC's). In one example, the controller 502 is in communication with, or operable to access or exchange signals with, a logic circuit 504. The logic circuit 504 includes at least one data storage or memory device. In this example, the memory device stores program code and instructions, executable by such processor, to control the stabilizing apparatus 100. In one example, such memory device includes: (a) RAM (MRAM); (b) ferroelectric RAM (FeRAM); (c) read only memory (ROM); (d) flash memory; (e) EEPROM (electrically erasable programmable read only memory); or a suitable combination of such memory devices. It should be appreciated that any other suitable magnetic, optical, or semiconductor memory may operate in conjunction with, or as part of, the stabilizing apparatus 100.

In one example, controller 504 is operatively connected to a plurality of input devices 506, including a plurality of buttons 508, and a plurality of output devices 510, including a gear assembly 512. In one example, the buttons 508, described below, include: (a) a Power button 514; (b) a Retract button 516; (c) a Extend button 518; and (d) a Lights button 520.

In one example, the Retract button 516 and the Extend button 518 enable the user to control the position of the latching mechanism. When the user actuates or presses the Retract button 516, the controller 504 causes the movable latches to move to the retracted position. In one example, when the user actuates or presses the Extend button 518, the controller 504 causes the movable latches to move to the extended position.

In one example, the Power button 506 enables the user to power on and off the stabilizing apparatus 100.

In one example, the Lights button 520 enables the user to power on and off the light attached to the end portion of the stabilizing apparatus 100.

In one example, the gear assembly 508 includes a first gear disposed inside the latching mechanism and a second gear operatively coupled to a movable latch. In this example, when a user presses the Extend or Retract button, the controller 504 causes the first gear to rotate. The first gear causes the second gear to rotate. As the second gear rotates, the movable latch operatively coupled to the first gear begins to either retract or extend depending upon the user input command. It should be appreciated that the above describe gear assembly would preferably include a first and second gear for each of the movable latches.

In one example, each of the plurality of movable latches 306 are operatively coupled to a spring loaded retraction mechanism. The spring-loaded retraction mechanism urges the plurality of movable latches 306 to have a predisposed retracted position.

In one example, each of the hook portions 332 are covered with a rubber or other material softer than metal.

The apparatus can be made from materials commonly associated with surgical devices. For example, the apparatus may be made of steel, plastic or stainless steel. The stabilizing mechanism may be disposable.

The present invention also includes a method for stabilizing an abdominal wall during a laparoscopic incision. The method includes partially inserting a latching mechanism of a stabilizing apparatus into a patient's navel. The latching mechanism includes a plurality of movable latches having hooks that latch to the patient's navel. The stabilizing apparatus further includes a moving mechanism to move the plurality of movable latches in an outward direction. The method further includes moving the movable latches in the outward direction to latch onto the patient's navel. The user of the apparatus then pulls the apparatus upwardly to stabilize the patient's abdominal wall.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A fascia stabilizing apparatus comprising:
a housing having a distal end;
a latching mechanism positioned at the distal end, the latching mechanism including a first latch having a first hook portion and a second latch having a second hook portion, the first latch and the second latch being movable between: (a) a first position which distances the first hook portion and the second hook portion from an elongated axis of the housing by a first distance; and (b) a second position which distances the first hook portion and the second hook portion from the elongated axis by a second greater distance; and
a graspable member operatively coupled to the first latch and the second latch, the graspable member operable to cause the first latch and the second latch to move between the first position and the second position.

2. The fascia stabilizing apparatus of claim 1, wherein the housing has an elongated tubular shape.

3. The fascia stabilizing apparatus of claim 1, wherein: (a) the housing includes an interior threaded surface; and (b) the latching mechanism includes a latch holder having a tubular portion, the tubular portion having an exterior threaded surface which matingly engages the interior threaded surface.

4. The fascia stabilizing apparatus of claim 1, wherein the latching mechanism includes a latch holder having: (a) a tubular portion; (b) a first curved extension which longitudinally extends from the tubular portion; and (c) a second curved extension which longitudinally extends from the tubular portion, wherein the first latch is disposed between the first curved extension and the second curved extension.

5. The fascia stabilizing apparatus of claim 1, wherein:
(a) the latching mechanism includes a latch holder having a tubular portion, the tubular portion defining a first opening; and
(b) the first latch including a top portion which defines a second different opening, wherein the first latch is pivotally connected to the tubular portion using a pin which is disposed in the first opening and the second opening.

6. The fascia stabilizing apparatus of claim 1, which includes an actuator operatively coupled to: (a) the graspable member; (b) the first latch; and (c) the second latch, wherein the actuator causes the first latch and the second latch to move between the first position and the second position when the graspable member is actuated.

7. The fascia stabilizing apparatus of claim 6, wherein: (a) the housing includes an interior threaded surface; and (b) the actuator includes a shaft having an exterior threaded surface which matingly engages the interior threaded surface.

8. The fascia stabilizing apparatus of claim 6, wherein:
(a) the actuator includes an elongated cylinder including: (i) a first top portion having a first top surface; and (ii) a bottom portion having a bottom surface and an angled surface;
(b) the first latch includes a second top portion having a second top surface and the second latch includes a third top portion having a third top surface;
(c) if the first latch and the second latch are each in the first position, the bottom surface of the elongated cylinder contacts the second top surface and the third top surface; and
(d) if the first latch and the second latch are each in the second position, the angled surface of the elongated cylinder contacts the second top surface and the third top surface.

9. The fascia stabilizing apparatus of claim 1, wherein the housing defines an interior space to enable a person to insert an auxiliary surgical instrument through said interior space.

10. The fascia stabilizing apparatus of claim 1, which includes a light attached to the distal end of the housing.

11. The fascia stabilizing apparatus of claim 1, which includes spring loaded retraction mechanism operatively coupled to the first latch and the second latch.
